(19) United States
(12) Patent Application Publication
(10) Pub. No.: US 2014/0207054 A1
(43) Pub. Date: Jul. 24, 2014

(54) APPARATUS AND METHOD OF TREATING A VEIN WITH THERMAL ENERGY

(71) Applicant: Veniti, Inc., St. Louis, MO (US)

(72) Inventors: Lowell S. Kabnick, Far Hills, NJ (US); Daniel Recinella, St. Charles, MO (US); Kimberly McCarthy, Ballston Spa, NY (US)

(73) Assignee: Veniti, Inc., St. Louis, MO (US)

(21) Appl. No.: 14/161,119

(22) Filed: Jan. 22, 2014

Related U.S. Application Data
(60) Provisional application No. 61/755,938, filed on Jan. 23, 2013.

(51) Int. Cl. A61B 18/04; A61M 5/14

(52) U.S. Cl. CPC A61B 18/042; A61M 5/14
USPC 604/26

(57) ABSTRACT

A method of delivering therapy to a treatment volume of a vein of a patient includes inserting a vapor delivery shaft into the vein at an entry location. The vein is fluidly coupled with other veins in a venous vasculature of the patient. The method also includes applying a compressive force to tissue surrounding the venous vasculature to compress and at least partially occlude the venous vasculature at an occlusion location other than the entry location. A treatment volume is formed between the entry location and the occlusion location. The method further includes delivering vapor to the vein through the vapor delivery shaft into the treatment volume.

### CROSS-REFERENCE TO RELATED APPLICATIONS

This application claims priority to U.S. Provisional Application No. 61/755,938, which was filed 23 Jan. 2013, and is entitled “Apparatus And Method Of Treating A Vein With Thermal Energy.” The entire disclosure of the 61/755,938 application is incorporated by reference.

### FIELD

This disclosure generally relates to treatment of blood vessel disorders. More specifically, this disclosure relates to using vapor therapy to reduce an inner diameter of a vessel in the leg of a patient.

### BACKGROUND

The human venous system of the lower limb consists essentially of the superficial venous system and the deep venous system with perforating veins connecting the two systems. The superficial system includes the great saphenous, small saphenous and collateral veins. The deep venous system includes the anterior tibial, peroneal, and posterior tibial veins which unite to form the popliteal vein, which in turn becomes the femoral vein and the common femoral vein when the deep femoral vein unites with the femoral vein. The small saphenous vein usually inserts into the popliteal vein, although may give rise to a cephalad extension terminating in the femoral vein or joining the intersaphenous vein terminating at the great saphenous vein. The great saphenous vein typically joins with the common femoral vein. At the level of the proximal thigh just peripheral to the saphenofemoral junction, the great saphenous vein gives off tributaries: epigastric, anterior and posterior thigh circumflex, external pudendal vein, and the circumflex iliac.

The venous systems contain numerous one-way valves for facilitating blood flow back to the heart. Venous valves are usually bicuspid valves, with each cusp, or leaflet, forming a sack or reservoir for blood which, under pressure, forces the free surfaces of the cusps together to prevent retrograde flow of the blood and allows antegrade flow to the heart. When an incompetent valve is in the flow path of retrograde flow toward the foot, the valve is unable to close because the cusps do not form a proper seal and retrograde flow of blood cannot be stopped.

Varicose veins occur as a result of weakening of the vein wall which then causes failure of the one-way valve system. This failure of the valve system allows venous reflux and can lead to venous hypertension. Venous hypertension may result in patient symptoms and clinical findings, such as varicose veins, skin discoloration, lipodermatosclerosis, and ulcers. Incompetence in the venous system can result from vein dilation, which causes the veins to swell with additional blood. As the veins swell, the cusps or leaflets of the venous valve at the commissure may separate. The leaflets are stretched by the dilation of the vein and concomitant increase in the vein diameter which the leaflets traverse. Stretching of the leaflets of the venous valve results in incompetent valves. Eventually the venous valve fails, thereby increasing the strain and pressure on the lower venous sections and overlying tissues.

The varicose vein condition includes dilatation and tortuosity of the superficial veins of the lower limb, resulting in unsightly protrusions, “heaviness” in the lower limbs, itching, pain, skin discoloration, lipodermatosclerosis, and ulceration. Varicose veins often involve incompetence of one or more venous valves, which allow reflux of blood from the deep venous system to the superficial venous system or reflux within the superficial system.

Current varicose vein treatments include invasive open surgical procedures such as vein stripping and occasionally vein grafting, venous valvuloplasty, and the implantation of various prosthetic devices. The removal of varicose veins from the body can be a tedious, time-consuming procedure, and the healing process can be painful and slow. Complications including scarring and the loss of the vein for future potential cardiac and other by-pass procedures may also result. Along with the complications and risks of invasive open surgery, varicose veins may persist or recur, particularly when the valvular problem is not corrected. Due to the long, arduous, and tedious nature of the surgical procedure, treating multiple venous sections can exceed the physical stamina of the physician, and thus render complete treatment of the varicose vein condition impractical.

Newer, less invasive therapies to treat varicose veins include intralumenal treatments to cause thermal damage to the endothelium in combination with shrinking of the collagen leading to thrombosis and fibrosis. This facilitates the collapse of the inner lumen. These therapies include foam sclerotherapy, as well as catheter, energy-based treatments such as laser, Radio Frequency (RF), or resistive heat (heater coil) treatments that effectively elevate the temperature of the vein wall to cause collagen contraction, an inflammatory response, and endothelial damage. Sclerotherapy, or delivery of a sclerosant directly to the vein wall, is typically not used with the larger trunk veins due to treatment complications of large migrating sclerosant boluses. Laser energy delivery can result in extremely high tissue temperatures which can lead to pain, bruising, and thrombophlebitis. RF therapy uses a heating coil or needle that can be ineffective due to inconsistent vein wall contact (especially in larger vessels). The catheter based treatments such as laser, resistive heater coil and RF energy delivery also typically require external vein compression to improve energy coupling to the vein wall. In addition, due to the size and/or stiffness of the catheter shaft and laser fibers and the heat generated (e.g., greater than 120° F.), none of these therapies are currently being used to treat tortuous surface varicosities or larger spider veins. The catheter or laser fiber will not transmit energy far enough because of the conduction methodology and distance between the energy source and the vein wall. They are currently limited in their use to large trunk veins such as the great saphenous vein. Tortuous surface varicosities are currently treated with sclerotherapy and ambulatory phlebectomy, while larger spider veins are currently only treated with sclerotherapy.

### SUMMARY

In an embodiment, a method of delivering therapy to a treatment volume of a vein of a patient is provided. The method includes inserting a vapor delivery shaft into the vein at an entry location. The vein is fluidly coupled with other veins in a venous vasculature of the patient. The method also includes applying a compressive force to tissue surrounding the venous vasculature to compress and at least partially occlude the venous vasculature at an occlusion location other than the entry location. A treatment volume is formed between the entry location and the occlusion location. The method further includes delivering vapor to the vein through the vapor delivery shaft into the treatment volume.

In an embodiment, a vapor delivery device is provided that includes a structural sheath and a vapor delivery shaft. The structural sheath has a hollow body with a distal end. The distal end is configured to be inserted into a vein of a patient at an entry location. The vein is fluidly coupled with other veins in a venous vasculature of the patient. The structural sheath is configured to structurally support the vein. The vapor delivery shaft is configured to be advanced through the structural sheath. The vapor delivery shaft has a tip that is positioned proximate to the distal end of the structural sheath within the vein. The vapor delivery shaft is configured to deliver vapor through the tip into a treatment volume of the vein. The treatment volume is defined between the entry location and an occlusion location other than the entry location where the venous vasculature is at least partially occluded due to a compressive force applied to tissue surrounding the venous vasculature

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is an embodiment of a vapor delivery catheter.

FIG. 2 shows another embodiment of a vapor delivery catheter with a sheath slightly retracted to reveal a vapor delivery tip.

FIG. 3 shows an embodiment of a vapor delivery catheter with a sheath slightly retracted to reveal a vapor delivery tip.

FIG. 4 shows an embodiment of a vapor delivery catheter with a sheath having windows or openings.

FIGS. 5A-5D illustrate a method of treatment of a vessel in accordance with an embodiment.

FIG. 6 illustrates a method of treatment of a vessel in accordance with an embodiment.

FIG. 7 illustrates a method of treatment of a vessel in accordance with an embodiment.

FIGS. 8A-8B illustrate a method of treatment of a vessel in accordance with an embodiment.

FIG. 9 is a flow chart for a method of delivering therapy to a vessel in accordance with an embodiment.

### DETAILED DESCRIPTION

The disclosure relates generally to systems and their methods of use to treat venous insufficiency. More particularly, the disclosed subject matter relates to vapor treatment of a vein to reduce its inner diameter to minimize and/or eliminate blood flow through the vein. The therapy is generally used to divert the flow of blood from an insufficient vein to a vein that is sufficient.

The vapor treatments described herein may be used to treat any vein, such as trunk vessels (e.g., a great or small saphenous vein), sub-truncal veins (e.g., accessory vessels) or spider veins. The veins treated may be varicose veins, although the treatments may be used to treat non-varicose veins. The disclosure is not, however, limited to the treatment of the veins and the anatomical locations of the veins that are described herein, such as veins in the leg region. For example, the invention may be used to treat veins outside the leg region, such as abdominal varicosities, hemorrhoids, varicoceles, and the like.

The treatments described herein generally include generating and delivering relatively high temperature (e.g., without limitation, greater than 37° C.) vapor through a delivery device to the lumen of a vein to reduce the inner diameter of the vein. A significant benefit of vapor is that it is mostly convection heating with some conduction versus the other devices which use a conduction delivery to close the vessel. Vapor flows to the internal surfaces of the vein due to the tendency of gases to expand to fill the volume of a defined space. Vapor delivery does not require external compression of the vein to enhance energy transfer from the delivery device to the vein wall, unlike other catheter-based treatments, such as laser, resistive heater coil, and RF energy delivery. Another significant benefit of the vapor delivery is the large amount of energy (e.g., 540 cal/g), released in the transition of the vapor into the liquid phase. A further significant benefit of the vapor is that it is self-limiting in temperature, as vapor ceases to conduct heat to the vessel wall once temperature equilibrium has been reached between the vapor and the vessel wall. This is unlike other treatments which continue to deliver thermal energy to the tissue to the point of extensive thermal injury.

The vapor (e.g., steam) may be generated in a variety of locations in the system. For example, the vapor may be generated in a remote boiler or control console separate from the delivery device, within a handle or handpiece, or within a portion of an elongate member (e.g., a catheter) that is inserted into the vein. The vapor may be generated in any portion of the elongate member that is either internal or external of the patient, for example. Vapor may be delivered to the targeted vein continuously or in pulses.

The vapor may propagate through open blood vessels in fluid communication with the vapor delivery catheter, including in veins where vapor treatment is not desired. The vapor propagation may be blocked by closed blood vessels. The vein treatment area may be modified or defined by closing access to venous areas where propagation of vapor is not desired. One way to close the vein lumen is to provide a compressive force to the vein area. The compressive force may cause the vein lumen to partially occlude and reduce the propagation of vapor past the occluded area. In some embodiments the compressive force may fully occlude or collapse the vein lumen and prevent propagation of vapor past the occluded or collapsed area. The reduction or prevention of vapor propagation may reduce or prevent damage to vein areas beyond the occlusion location while treating the targeted treatment area.

The targeted vein areas for receiving vapor treatment from the vapor delivery catheter may be defined by applying pressure to partially occlude or collapse areas of the vein where vapor treatment beyond the occluded or collapsed areas is not desired. The treatment volume of a treated vein may be the space within the vein between the partially occluded or collapsed area of the vein and a tip of the vapor delivery catheter. Optionally, the treatment volume may extend from the partially occluded or collapsed area of the vein beyond the tip of the catheter to a vein entry point of the catheter (e.g., through which the catheter enters the vein). The targeted vein area may be treated in multiple segments or treatment volumes. For example, a compressive force may be applied to a first location of the vein to establish a first treatment volume. Vapor may be delivered to the first treatment volume to treat the vein. After delivering vapor to the first treatment volume, the vapor delivery catheter may be pulled back a desired distance along the same vein. A new treatment volume may be established by applying a compressive force to a new location on the vein followed by vapor delivery to the new treatment volume. Pulling back on the vapor delivery catheter and applying a compressive force to partially occlude or collapse the vein where vapor was previously delivered or where treatment is not desired may be repeated until the entire targeted vein area has been treated. The method allows for segmental ablation of the vessel.

The vapor delivered to each treatment volume expands to fill the vein and contact the walls of the vein. The vapor delivered to the vein may treat the entire length of the vein in the treatment volume (e.g., up to about 15 cm or more of the vein). RF and laser methods are slower as they require a slow pullback on the catheter and only treat the area of the vein that is adjacent to the energized portion of the catheter. Vapor delivery to the treatment volume allows for faster and improved vein treatment than prior art methods such as laser treatment and RF delivery. The vapor treatment of multiple treatment volumes or vein segments results in a more consistent thermal energy delivery and a more uniform vein treatment.

Vapor treatment may also treat narrow areas of the vein where laser and RF treatments will not work. For example, the vapor treatment may be used to treat vein side branches and tortuous veins that are not accessible to RF and laser catheters.

The vapor and condensed liquid (formed by the vapor condensing) may provide heat transfer to the treated vein or veins in the form of convective heating and/or conductive heating. For example, the vapor may provide mostly convective heating, and the condensed liquid may provide mostly conductive heating. The use of compression may limit the passage of vapor to other areas of the vein, minimizing heat transfer to the other areas of the vein. Minimizing the convective and conductive heat transfer to areas of the vein where heat transfer is undesirable limits injuries and damage to those tissue areas.

An example of an area where vapor propagation is undesirable is within the femoral veins. Vapor introduced to the saphenous veins may propagate past the Sapheno Femoral Junction (SFJ) or saphenopopliteal junction (SPJ) to the deeper femoral veins. Accidental treatment or vapor exposure to the deep vein may ablate, shrink, or collapse the deep vein. Treatment of the deep veins is undesirable because it could cause an outflow obstruction as well as deep venous thrombosis, which may lead to pulmonary embolism. Compression may be used to prevent propagation of vapor past the SFJ or SPJ to the deep veins. In some embodiments, occlusion of the vein at or adjacent to the SFJ may be verified visually or automatically using intravascular ultrasound imaging.

A compressive force may be used to create the desired treatment volume or space. For example compression may be applied to one or more areas of the vein to reduce or block vapor propagation to those vein areas. As the vapor treatment progresses, compression may be applied to additional areas of the vein to reduce or block vapor propagation as desired. Optionally, the vein may be compressed by compressing the tissue around the vein area.

In some embodiments, the compressive force may be applied to the leg of the patient to partially occlude, fully occlude, or collapse portions of the leg veins that are not to be exposed to or treated by vapor. In some embodiments the area around the SFJ may be compressed such that the vein is partially or fully occluded. Vapor is prevented from propagating past the SFJ to the deep vein blood vessels when the SFJ is in an occluded state. In some embodiments an area of the vein between the SFJ and the vapor delivery device may be compressed. For example, the great saphenous vein may be compressed about 5 cm away from the SFJ towards the vapor delivery device. In another example, the great saphenous vein may be compressed about 10 cm away from the SFJ towards the vapor delivery device. Compressing the great saphenous vein about 10 cm away from the SFJ may also prevent vapor propagation to some of the feeder veins.

The compressive force may be applied throughout the treatment of the vein or periodically. In an embodiment, the compression may be applied to the area around the SFJ during the beginning of the treatment and then stopped after the vein has collapsed in the area adjacent to the compressed area around the SFJ. In some embodiments, the treated collapsed area may reduce or prevent propagation of vapor past the SFJ to the deep veins.

In some embodiments the compressive force may be selectively applied based on the targeted region of the vein. For example, the compressive force may be applied to partially occlude or collapse branches or portions of veins where treatment with vapor is not desired.

The compressive force may be applied in an amount sufficient to compress superficial veins. The compressed vein may be partially occluded or collapsed. The compressive force may be applied by a doctor or medical professional. The compressive force may be applied manually by hand or by using a medical tool or device. Selective partial or complete compression of tributaries at varying, progressive locations may be used to gradually close the tributaries to guide the vapor through the tributaries to deliver treatment.

The status of the vein may be confirmed after the compressive force is applied. For example, ultrasound imaging may be used to confirm that the vein is partially occluded or collapsed in the area where the compressive force is applied.

As described above, compression may be used to partially occlude or collapse areas of veins where vapor treatment is undesirable. Compression of the vessel or movement of the vein wall into the lumen in the targeted vein areas prior to and/or during treatment is not desired. This compression or movement may be due to: administration of perivenous anesthesia and the fluid volume delivery and associated needle stick; administration of anesthetic or cooling fluid (typically 0.9% normal saline) around or on top of the vein; ultrasound probe pressure; or vein spasm (due to irritation of the vein due to catheter placement; cold procedure room; needle stick for local anesthetic; or cold saline drip from catheter tip). According to some embodiments, such vessel wall movement, luminal diameter reduction or distortion (e.g. flattening), or full lumen collapse (e.g., via administration of perivenous anesthesia) will not allow the vapor to freely flow from vapor catheter tip exit ports out to the full internal luminal surface of the vein. In some instances, full lumen collapse over the catheter tip exit ports may completely block and prevent the delivery of vapor to a collapsed vessel. Therefore, preventing vessel collapse in the targeted area and administering symmetrical and consistent vapor energy to the full internal luminal surface of the vein in the targeted area is desirable to achieve proper vein shrinkage of the targeted areas of the veins. In some embodiments the vapor delivery catheter may be used with a structural sheath to reduce vessel wall movement and reduction in the diameter of the lumen of the vein areas targeted by vapor delivery.

Although a structural sheath is shown in some of the figures, the use of the structural sheath with the vapor delivery catheter is optional. In some embodiments the vapor delivery catheter is inserted into the blood vessel without using a structural sheath.

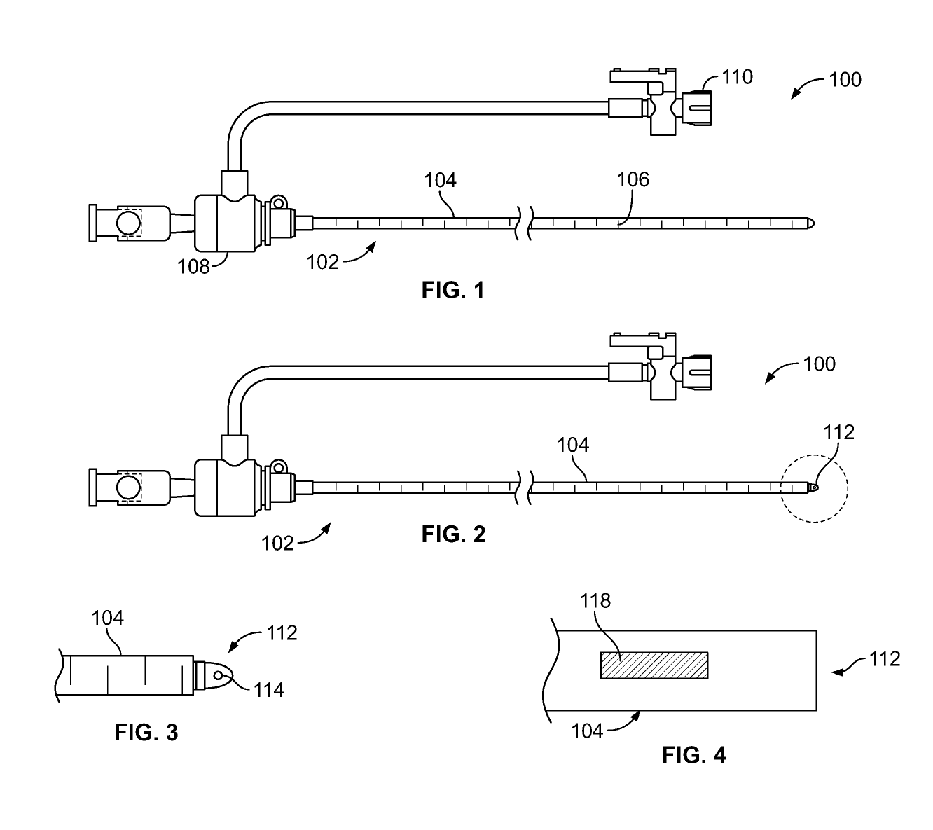

FIG. 1 illustrates a vapor catheter 100 configured to prevent luminal space reduction from occurring during vapor therapy treatment of blood vessels. In FIG. 1, the catheter 100 includes an elongated catheter shaft 102 and a structural sheath 104 disposed over the shaft. The sheath may be retractable from the catheter shaft 102 to reveal a vapor delivery tip (not shown in FIG. 1). The sheath 104 may include markings 106 to facilitate the determination and control of pull-back length and timing. In some embodiments, the markings may be spaced apart by a known distance (e.g., about 1 cm). The markings may further include numbering and/or lettering. The sheath 104 is configured to have a structural strength sufficient to hold its shape and prevent collapse of the vessel (e.g., support the vessel) during or after application of perivenous anesthesia.

The catheter 100 may further include a valve 108 and a flush port 110. The valve 108 may be configured to couple the catheter 100 to a control system and/or a vapor source. In some embodiments, the catheter 100 receives vapor from an external vapor source (e.g., a remote boiler). In other embodiments the catheter 100 generates vapor within the catheter 100 itself. The flush port 110 may facilitate flushing the catheter 100 with, for example, saline or another fluid/gas prior to or after therapy.

FIGS. 2-3 illustrate one embodiment of the vapor catheter 100 with the structural sheath 104 slightly retracted proximally from the catheter shaft 102 to reveal a portion of a vapor delivery tip 112. In some embodiments, the sheath 104 may be pulled back and locked in place to expose only the tip 112 of the vapor catheter 100. FIG. 2 shows the catheter 100 with the sheath 104 slightly retracted, and FIG. 3 is a close-up view of the tip 112 revealing vapor port(s) 114. In the embodiment of FIGS. 2-3, the sheath 104 may be a heat resistant sheath. Examples of heat resistant sheath materials include polymers and other plastics with high heat tolerance.

In the embodiment illustrated in FIGS. 2-3, the tip 112 of the structural sheath 104 may be positioned near the vapor ports 114 of the vapor catheter 100 to prohibit vessel walls from collapsing around the vapor ports 114 when perivenous anesthesia is applied to the patient. By preventing the collapse of the vessel walls around the vapor catheter 100, vapor is allowed to escape from the vapor ports 114 to treat the vessel.

According to the embodiment of FIG. 4, a structural sheath 104 may include windows, holes, openings, or ports 118 to further allow propagation of vapor from the vapor delivery catheter 100 to the vessel walls. In this embodiment, the window 118 is shown as a rectangular window in the structural sheath 104. However, in other embodiments, other types, sizes, and shapes of windows or ports may be used as long as the windows or ports are configured to allow vapor to propagate through the structural sheath 104 and into the vessel walls. The vapor delivery catheter 100 may be the same length as the structural sheath 104. The vapor delivery tip 112 of the vapor catheter 100 may be disposed at or near an opening of the sheath 104 at a distal end of the sheath 104. When vapor is delivered from the vapor catheter 100, the vapor may propagate through the window 118 of the sheath 104 and also through the opening in the sheath 104.

Methods of using the vapor delivery catheter 100 described above will now be discussed. The specific clinical steps to be used are included for illustration purposes and are not specific constraints of this disclosure. A micro introducer kit (not shown) may be used to gain venous access to a vein of a patient. The micro introducer kit may include a needle, a guide wire, and a microintroducer. For example, the micro introducer kit may include a 19 or 21 gauge needle and a 0.018 inch diameter guide wire. Next, a larger diameter guide wire (e.g., 0.035 inch guide wire) may be advanced to the SFJ. Ultrasound imaging may be used to verify the location of the SFJ. The location of the micropuncture and guide wire in the blood vessel may also be verified using ultrasound imaging. In some embodiments compression may be applied to an area of the blood vessel upstream of the introducer kit or guidewire, and intravascular ultrasound may be used to confirm if the blood vessel is a vein or an artery. Typically, a vein partially occludes or collapses in response to the compression, whereas an artery does not collapse in response to the compression.

Referring to FIG. 1, the structural sheath 104 may be inserted into the vein to be treated to the point at which the treatment is to be initiated. Next, perivenous anesthesia may be administered to the patient. Cooling fluid, such as saline, may be applied to the vein or around the vein if desired. The structural sheath 104 is configured to have sufficient structural strength to “stent” open the vein to prohibit vasospasm luminal reduction or other luminal reduction during the administration of perivenous anesthesia or cooling fluid around the vein circumference. It is desirable for the sheath to prevent the vein from collapsing during the administration of anesthesia, in order to allow for vapor delivery in a subsequent step.

After administering the anesthesia, the catheter shaft 102 may be inserted into the sheath 104 and advanced until the tip 112 (shown in FIG. 2) of the catheter 100 is even with the end of the sheath 104. After the location of the sheath 104 and catheter tip 112 are confirmed (e.g., via ultrasound), a distal end of the sheath 104 is retracted sufficiently to expose the vapor ports 114 as shown in FIG. 2. A compressive force may be applied to an area on the vein upstream or downstream of the vapor ports 114 to partially occlude or collapse the area of the vein where vapor treatment is not desired. For example, the SFJ or the saphenous vein at a distance from the SFJ may be at least partially occluded by the compressive force to prevent vapor from propagating to the SFJ and into any deep veins.

After inserting the catheter shaft 102, vapor energy may delivered from the catheter shaft 102 through the vapor ports 114 to the vein to be treated. The vapor energy may be generated in a remote boiler, in a handle connected to the catheter 100, or within the catheter 100 itself, for example. The vapor may be pulsed or continuously delivered. During the vapor treatment, the treatment volume of the vein may be defined by providing a compressive force to occlude one or more vein areas a distance from the vapor ports 114. Vapor is supplied to the treatment volume. After a first treatment volume is treated, the catheter 100 is pulled back a desired distance and another compressive force is applied at a distance from the new position of the vapor ports 114 to define a second treatment volume. Vapor is supplied to the second treatment volume. The steps may be repeated until the desired length of the vein is treated. The structural sheath 104 is configured to prohibit compression or movement of the vessel before and during vapor therapy to allow the vapor to contact the full internal luminal surface of the vein areas to be treated, thereby providing symmetrical and consistent application of vapor energy to the vein to be treated.

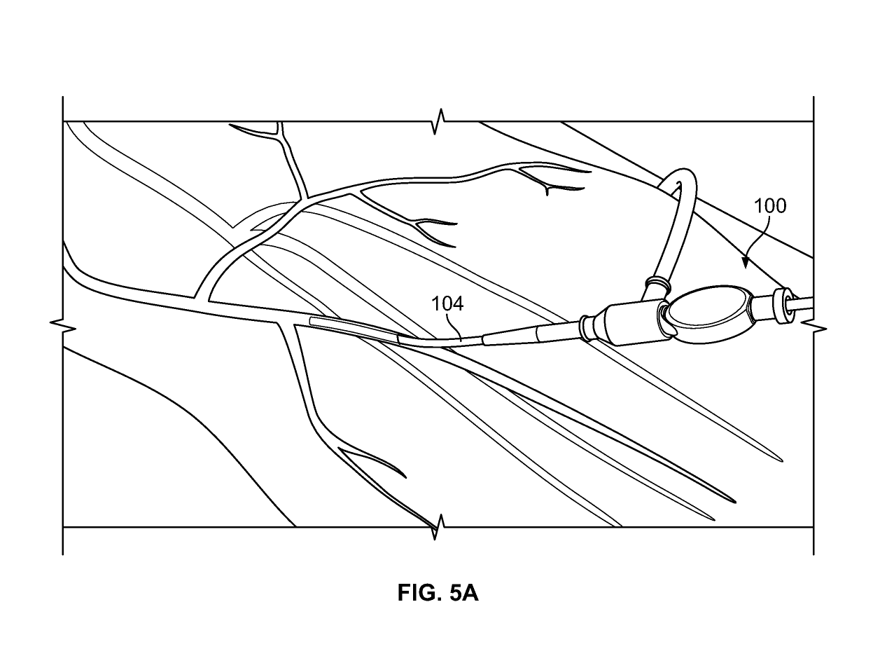

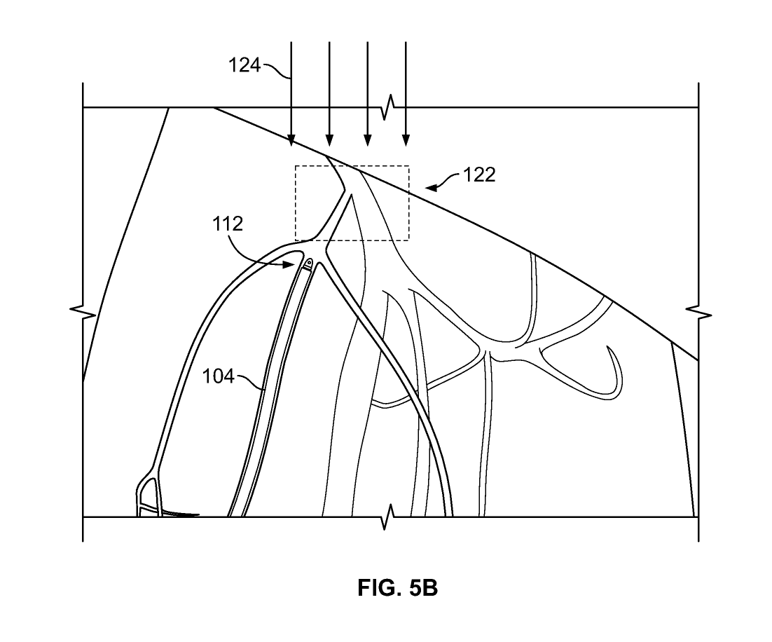

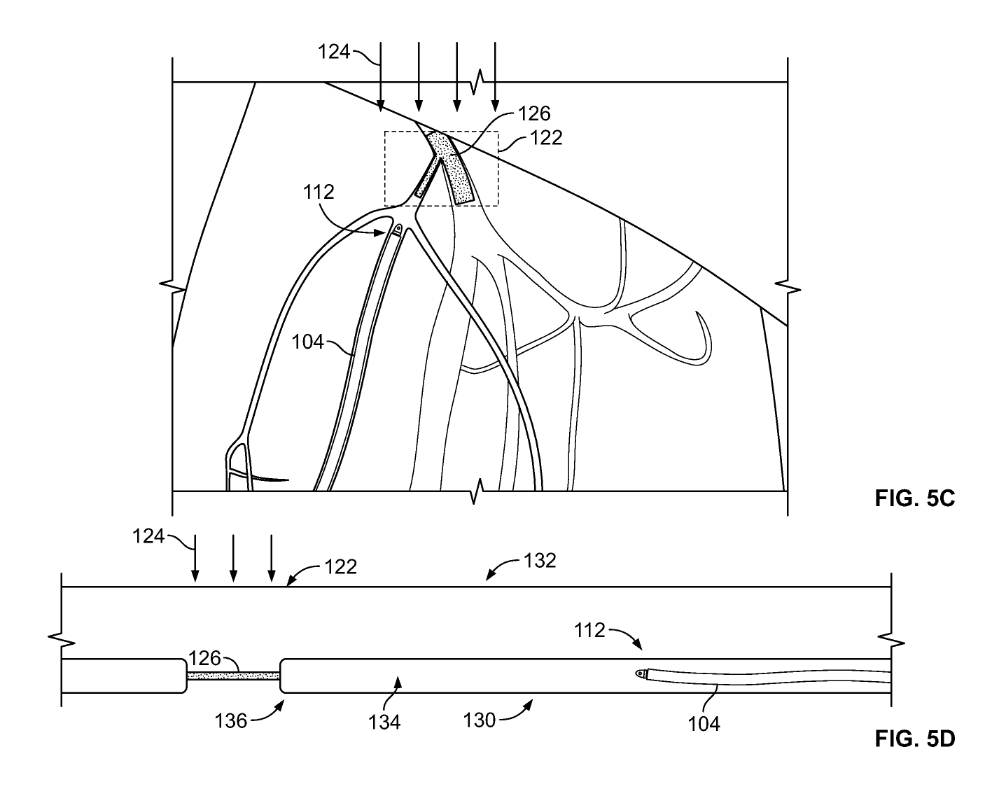

FIGS. 5A-5D illustrate a method of delivering therapy to a vein according to one embodiment. FIGS. 5A-5D show the use of a structural sheath 104. In an alternative embodiment, the catheter 100 may be used without the structural sheath 104. Referring to FIG. 5A, the structural sheath 104 may be inserted into an access point of a vessel to be treated. In one embodiment, the access point for the sheath 104 may be the great saphenous vein (GSV) in the leg of a patient. In another embodiment, the access point may be the small saphenous vein, for example.

Referring to FIG. 5B, the structural sheath 104 may be advanced along the vessel to be treated to the point where therapy is to be applied. As used herein, “central” is defined as towards the heart and “peripheral” is towards an extremity, such as a foot. In one embodiment, the delivery tip 112 is positioned peripheral to the sapheno femoral junction (SFJ) at a distance of up to approximately 5 cm from the SFJ. Positioning may be verified using ultrasound imaging. A compressive force 124 may be applied to the leg to collapse the vein in an area where vapor propagation is not desired. For example, compressive force 124 may be applied to a region 122 including the SFJ to compress the vein at the junction, thereby preventing the propagation of vapor to the SFJ and the deep veins beyond the SFJ. As shown in FIG. 5C, the compressive force 124 is applied to region 122 to occlude a portion of the vein in area 126 around the SFJ. The occluded area or location 126 around the SFJ blocks the vapor from propagating to the SFJ thereby preventing propagation of vapor to the deep veins.

FIG. 5D is a schematic cross sectional view of a vein 130. The structural sheath 104 and vapor delivery tip 112 of the vapor catheter 100 (shown in FIG. 1) are located in the vein 130. The compressive force 124 may be applied to the skin 132 at region 122. The compressive force 124 may cause the vein 130 to partially occlude or collapse at area 126. The partially occluded or collapsed area 126 may define a treatment volume 134 between the vapor delivery tip 112 and the occlusion point 136. During treatment the vapor expands from the vapor delivery tip 112 to occlusion point 136, which is the peripheral side of the occluded area 126. The occluded area 126 prevents propagation of vapor to the central side of the occlusion location. The treatment volume 134 may include the length of the vein 130 between the vapor delivery tip 112 and the occlusion point 136. Vapor may also expand between the vapor delivery tip 112 in the peripheral direction to the vapor catheter entry point (not shown) into the vein 130, such that the vapor treatment volume 134 includes the space between the vapor delivery tip 112 and the vapor catheter entry point into the vein 130.

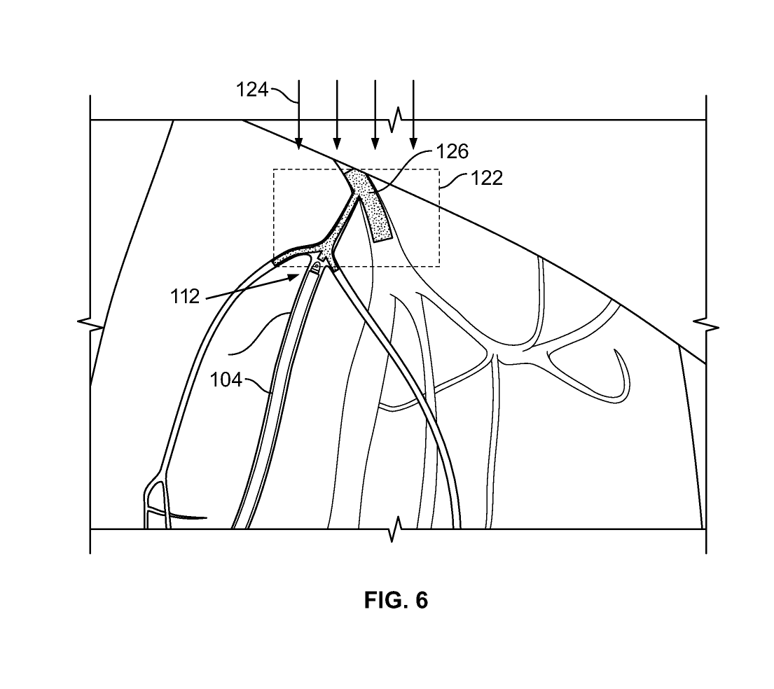

Referring to FIG. 6, the structural sheath 104 may be advanced along the vessel to be treated to the point where therapy is to be started (e.g., delivered). In one embodiment, the delivery tip 112 is positioned peripheral to the SFJ and locations where the feeder veins meet the great saphenous vein. In some embodiments, the delivery tip 112 may be positioned about 10 cm from the SFJ to insure that the SFJ and feeder veins are central to the delivery tip 112. The compressive force 124 may be applied to region 122, with the region 122 including the SFJ and locations where the feeder veins join the great saphenous vein, to compress the veins and prevent propagation of vapor to the SFJ, feeder veins, and the deep veins past the SFJ. The compressive force 124 is applied to region 122 to occlude a portion of the vein in area 126 around the SFJ, great saphenous vein, and feeder veins. The occluded area 126 around the SFJ blocks the vapor from propagating to the SFJ thereby preventing propagation of vapor to the deep veins. The occluded area 126 also blocks the propagation of vapor to the feeder veins.

In some embodiments, the start point for placement of the delivery tip 112 varies based on the condition of the vein to be treated. Proper positioning of the vapor catheter 100 may be confirmed with ultrasound imaging, for example. The vapor catheter 100 may be inserted into the sheath 104 and advanced towards the opening at the distal end of the sheath 104. The vapor delivery ports 114 of the catheter 100 may be advanced beyond the opening of the structural sheath 104, as described above, to expose the vapor delivery ports 114 to the interior of the vein for vapor delivery.

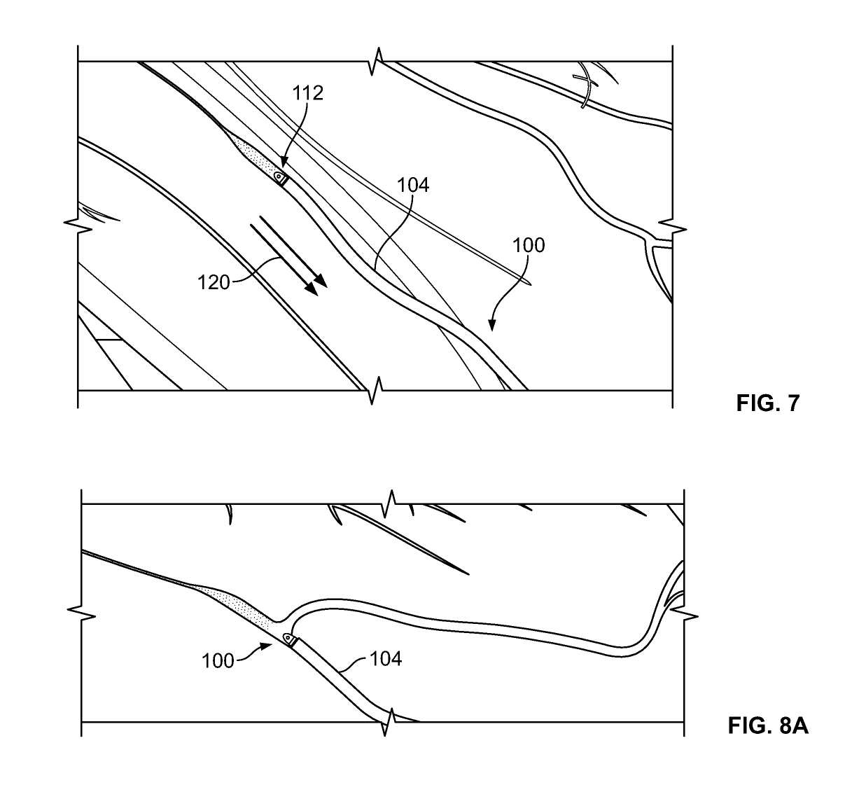

In some embodiments perivenous anesthesia may be applied in or around the vessel to be treated, as known. The structural sheath 104 is configured to prohibit collapse of the vessel due to the application of the perivenous anesthesia, or due to spasm of the vein. Referring now to FIG. 7, vapor may be delivered from vapor delivery tip 112 to the vessel to be treated. The vapor may propagate out through the vapor catheter 100. The vapor may expand within a treatment volume of the vein that is defined at one end by a partially occluded or collapsed portion of the vein caused by a compressive force and at the other end by the vapor catheter 100. After treating a first treatment volume of the vein, the catheter may be pulled peripherally along the length of the vessel to be treated (e.g., in direction 120), or pulled along at least a portion of the vessel that requires luminal diameter reduction. After pulling the catheter peripherally along the length of the vessel a second treatment volume may be defined by applying a second compressive force to an area of the vein. Vapor may be delivered to the second treatment volume of the vein. The catheter may then be further pulled along the length of the vein and additional portions of the vein may be treated.

Delivering vapor, pulling the catheter, and applying compressive force may be repeated to treat the vein until the desired treatment areas have been treated. This method provides for segmental ablation of the vessel. The rate of pulling may be determined based on the size of the vessel, blood flow rate in the vessel, fluid in the vessel, and/or the amount of vapor energy needed to treat the volume of the vessel. As shown, vapor delivery reduces the diameter of the treated vessel. The structural sheath 104, however, prevents collapse of the vein onto the catheter 100 prior to and during vapor delivery. In some embodiments the structural sheath 104 is optional and the treated vein area is defined by selectively collapsing portions of the vein by applying pressure to the veins to create treatment segments. The applied pressure is central to the catheter tip and the sheath. In some embodiments the treatment segments or volumes include about 5 cm of the vein length central to the catheter tip 112. In some embodiments the movement of the catheter 100 is optional and the vein treatment area is defined by selectively collapsing different portions of the vein by applying pressure to different regions along the veins.

Pressure (e.g., a compressive force) may be applied to region 122 to prevent vapor propagation throughout the treatment process. In some embodiments the pressure may be applied to region 122 at the beginning of the process to partially occlude or collapse area 126. The vapor treatment may collapse a portion of the vein between the occluded area 126 and the tip 112 of the vapor delivery device. The collapsed portion from the vapor treatment may prevent or greatly reduce the propagation of vapor to the SFJ. In some embodiments the pressure (e.g., compressive force) is applied at the beginning of the treatment and later stopped after enough of the superficial vein has been collapsed by the treatment to minimize or prevent the propagation of vapor past the collapsed treated area to the SFJ.

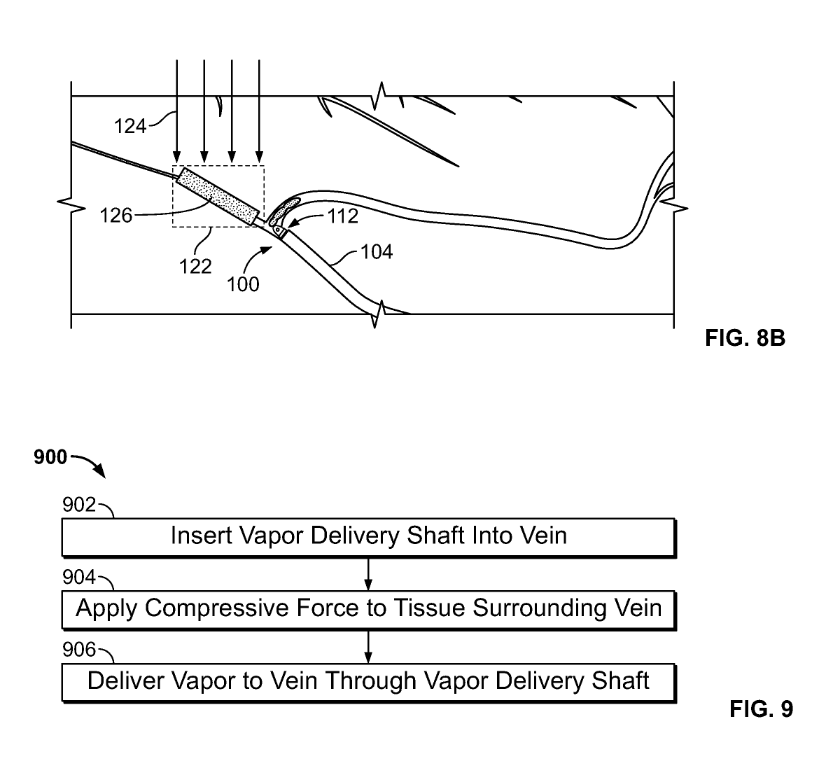

FIGS. 8A-8B illustrate another embodiment of the method, useful, for example, in the treatment of feeder veins stemming from larger vessels in the leg. In some embodiments, it may be desirable to treat feeder veins with vapor therapy alongside treatment of the main vessel. As described above, the vapor catheter 100 may be pulled peripherally along the vessel to be treated (such as the GSV) to deliver vapor to segmented treatment volumes of the vessel. When the catheter reaches the junction of the main vessel and a feeder vein, compressive force 124 may be applied to facilitate vapor propagation into the feeder vein and to deliver vapor into the feeder vein, as shown in FIG. 8B. This may be accomplished, for example, by selectively applying compression to the vein to channel the vapor into the feeder vein. Vapor may be delivered into the feeder vein to treat the vein by reducing the diameter of the vein as shown in FIG. 8B. The amount of time to deliver vapor to the feeder vein may vary based on the size/diameter of the feeder vein. In some embodiments a compressive force may be applied to the feeder vein to partially or fully occlude any portions of the feeder vein where vapor treatment is not desirable.

The length of vein in the treatment volume may vary based on a number of factors including the varicose vein length, vein volume, blood flow rate, pressure in the vein, presence of other fluids like perivenous anesthesia, etc. In some embodiments the length of vein in the treatment volume may be about 1 cm or greater. In some embodiments the length of vein in the treatment volume may be less than about 15 cm. In some embodiments the length of vein in the treatment volume may be about 1 cm to about 45 cm. In some embodiments the length of vein in the treatment volume may be about 1 cm to about 15 cm. In some embodiments the length of vein in the treatment volume may be about 1 cm to about 10 cm. In some embodiments the length of vein in the treatment volume may be about 1 cm to about 7 cm. In some embodiments the length of vein in the treatment volume may be about 1 cm to about 5 cm. In some embodiments the length of vein in the treatment volume may be about 1 cm to about 3 cm. In some embodiments the length of vein in the treatment volume may be a distance from about the entry site to about the SFJ.

The treatment time for each treatment volume may vary based on a number of factors, such as vein length in the treatment volume, vein volume in the treatment volume, pulsed or continuous vapor (e.g., steam) delivery, blood flow, other fluids in the vein, desired level of ablation, vapor flow rate, etc. In some embodiments the treatment time is on the order of a few seconds, such as about 0.1 seconds to about 60 seconds.

FIG. 9 is a flow chart for a method 900 of delivering therapy according to an embodiment. The therapy may be delivered to a treatment volume of a vein of a patient. The vein is fluidly coupled with other veins in a venous vasculature of the patient. At 902, a vapor delivery shaft is inserted into the vein at an entry location.

At 904, a compressive force is applied to tissue surrounding the venous vasculature (e.g., including the vein) to compress and at least partially occlude the venous vasculature at an occlusion location. The compressive force may be applied to the tissue surrounding an area of the vein (e.g., of the venous vasculature) where vapor treatment is to be avoided. The compressive force may be applied using a hand and/or a surface of an ultrasound imaging device.

The occlusion location is other than (e.g., spaced apart from) the entry location of the vapor delivery shaft. Optionally, the occlusion location may be at (e.g., includes), adjacent to, and/or peripheral to a Sapheno Femoral Junction (SFJ) of the patient. The occlusion location may be a peripheral length of the vein of about 10 cm or less. Optionally, ultrasound imaging may be used to confirm that the vein of the venous vasculature at the occlusion location is at least partially occluded after applying the compressive force.

The occlusion location at least partially defines a treatment volume of the vein. The treatment volume is the space within the venous walls of the vein along a length of the vein between the occlusion location and the entry location of the vapor delivery shaft. Optionally, the treatment volume may be defined along a length of the vein between the occlusion location and a tip (e.g., a vapor delivery tip) of the vapor delivery shaft, instead of the entry location. The occlusion location may be central to the tip of the vapor delivery shaft. The treatment volume may extend a length of the vein of about 1 cm to about 10 cm.

At 906, vapor is delivered to the vein through the vapor delivery shaft into the treatment volume. The vapor within the treatment volume provides treatment in the form of thermal energy to the venous walls of the vein within the treatment volume. The vapor may be delivered through the tip (e.g., vapor delivery tip) of the vapor delivery shaft. Optionally, the vapor may be generated remotely (e.g., at a remote location) from the vapor delivery shaft. Alternatively, the vapor may be generated within the vapor delivery shaft itself. At least some of the vapor may be blocked from propagating centrally beyond the occlusion location by the at least partially occluded venous vasculature due to the compressive force.

Optionally, prior to inserting the vapor delivery shaft into the vein at 902, a structural sheath, configured to structurally support the vein, may be inserted into the vein. The vapor delivery shaft may be inserted into the vein by advancing the vapor delivery shaft into the structural sheath until a tip (e.g., vapor delivery tip) of the vapor delivery shaft is positioned proximate to a distal end of the structural sheath. Prior to delivering the vapor to the vein, the distal end of the structural sheath may be retracted peripherally along the vapor delivery shaft to expose the tip.

After delivering the vapor at 906, optionally the vapor delivery shaft may be pulled peripherally along the vein. The vapor delivery shaft may be pulled along a length of the vein to be treated a distance of about 1 cm to about 10 cm. After pulling the vapor delivery shaft, another compressive force may be applied to different tissue surrounding the venous vasculature to compress and at least partially occlude the venous vasculature at an updated occlusion location that is other than the entry location and the previous occlusion location. A new treatment volume is formed between the entry location and the updated occlusion location. Vapor is delivered to the vein through the vapor delivery shaft into the new treatment volume. The method 900 may further include repeating the steps that include pulling the vapor delivery shaft, applying a compressive force, and delivering vapor, until a desired length of the vein is treated.

### Example 1

### 40 cm Refluxing Vein GSV

Using lidocaine, a few cc are infiltrated at the intended entry site. A 21 gauge needle is inserted into the targeted vein and a 0.018 inch guide wire is passed through the needle and into the vein. After the entry needle is removed, the micropuncture sheath is placed over the wire and pushed into the vein. Next, the 0.018 inch guide wire and dilator are removed from the micropuncture sheath and a 0.035 inch wire is inserted into the micropuncture sheath and positioned into the common femoral vein by ultrasound guidance. The micropuncture sheath is removed and a 7 Fr sheath is then introduced into the vein over the wire and placed by ultrasound guidance about 5-10 cm peripheral to the saphenofemoral junction (SFJ). After removing the dilator of the sheath, the vapor delivery catheter is placed into the sheath and directed to the end of the sheath.

The sheath is then pulled back exposing the vapor delivery portion of the catheter. The sheath engages the catheter in a locking position. The positioning of the sheath and vapor delivery catheter is re-verified using duplex ultrasound. Perivenous anesthesia, dilute local, is inserted along the entire length of the vein to be treated. In addition to the numbing action, perivenous anesthesia is used to protect surrounding structures. Perivenous anesthesia is controlled by ultrasound guidance to surround the targeted vein from the entry site to the SFJ. The vapor delivery catheter is then attached to the machine and activated according to the manufacturer's recommendation.

Once the vapor delivery tip is rechecked by ultrasound, compression using a digital ultrasound probe is applied to the great saphenous vein about 5 cm from the SFJ. The compression occludes the vein central to the tip of the catheter and compartmentalizes the central area preventing central vapor convection and establishes a treatment volume between the occlusion point and the vapor delivery device. The vapor delivery device is activated and vapor is provided to the vein treatment volume for a period of time. After treatment of the first treatment volume the catheter and sheath is pulled back about 5 cm to 7 cm. Compression is applied to the vein in an area adjacent to the previous location of the tip of the vapor delivery catheter to establish a second vein treatment volume. This method of segmental ablation is repeated along the length of the vein. Establishing a successive occlusion point about 5 cm to about 7 cm from the previous occlusion point prevents vapor expansion into prior-treated areas, and thus prevents retreatment of treated areas. This prevents possible perforation and sequela as a result. Once the treatment is finished, both the vapor catheter and sheath are removed. Steri-strips are applied to the insertion site, covered with a dry sterile dressing. Finally, the leg is placed into a graduated compression stocking.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A method of delivering therapy to a treatment volume of a vein of a patient, comprising:
inserting a vapor delivery shaft into the vein at an entry location, the vein fluidly coupled with other veins in a venous vasculature of the patient;
applying a compressive force to tissue surrounding the venous vasculature to compress and at least partially occlude the venous vasculature at an occlusion location other than the entry location, forming a treatment volume between the entry location and the occlusion location; and
delivering vapor to the vein through the vapor delivery shaft into the treatment volume.

2. The method of claim 1, wherein the vapor is generated remotely from the vapor delivery shaft.

3. The method of claim 1, wherein the vapor is generated within the vapor delivery shaft.

4. The method of claim 1, further comprising:
inserting a structural sheath into the vein prior to inserting the vapor delivery shaft into the vein, the structural sheath being configured to structurally support the vein, wherein inserting the vapor delivery shaft into the vein includes advancing the vapor delivery shaft into the structural sheath until a vapor delivery tip of the vapor delivery shaft is positioned proximate to a distal end of the structural sheath.

5. The method of claim 4, further comprising, prior to delivering the vapor, retracting the distal end of the structural sheath peripherally along the vapor delivery shaft to expose the vapor delivery tip.

6. The method of claim 1, wherein the treatment volume includes a length of the vein between the occlusion location and a tip of the vapor delivery shaft of about 1 cm to about 10 cm.

7. The method of claim 1, further comprising:
after delivering the vapor, pulling the vapor delivery shaft peripherally along the vein;
applying another compressive force to different tissue surrounding the venous vasculature to compress and at least partially occlude the venous vasculature at an updated occlusion location other than the entry location, thereby forming a new treatment volume between the entry location and the updated occlusion location; and
delivering vapor to the vein through the vapor delivery shaft into the new treatment volume.

8. The method of claim 7, wherein pulling comprises pulling the vapor delivery shaft along a length of the vein to be treated a distance of about 1 cm to about 10 cm.

9. The method of claim 7, further comprising repeating pulling the vapor delivery shaft, applying a compressive force, and delivering vapor until a desired length of the vein is treated.

10. The method of claim 1, wherein the occlusion location is at least one of at, peripheral to, or adjacent to a Sapheno Femoral Junction (SFJ) of the patient.

11. The method of claim 1 wherein the occlusion location is central to a vapor delivery tip of the vapor delivery shaft, the vapor to the vein delivered through the vapor delivery tip, the vapor blocked from propagating centrally beyond the occlusion location by the at least partially occluded venous vasculature due to the compressive force.

12. The method of claim 1, further comprising confirming that the vein of the venous vasculature at the occlusion location is at least partially occluded after applying the compressive force using ultrasound imaging.

13. The method of claim 1, wherein the compressive force is applied to the tissue surrounding an area of the vein where vapor treatment is to be avoided.

14. The method of claim 1, wherein the occlusion location includes the Sapheno Femoral Junction (SFJ) and a peripheral length of the vein of about 10 cm or less.

15. The method of claim 1, wherein the compressive force is applied using at least one of a hand or a surface of an ultrasound imaging device.

16. A vapor delivery device comprising:
a structural sheath having a hollow body with a distal end, the distal end configured to be inserted into a vein of a patient at an entry location, the vein fluidly coupled with other veins in a venous vasculature of the patient, the structural sheath configured to structurally support the vein, and
a vapor delivery shaft configured to be advanced through the structural sheath, the vapor delivery shaft having a tip that is positioned proximate to the distal end of the structural sheath within the vein, the vapor delivery shaft configured to deliver vapor through the tip into a treatment volume of the vein, the treatment volume defined between the entry location and an occlusion location other than the entry location where the venous vasculature is at least partially occluded due to a compressive force applied to tissue surrounding the venous vasculature.

17. The vapor delivery device of claim 16, wherein the distal end of the structural shaft is configured to retract peripherally along the vapor delivery shaft to expose the tip of the vapor delivery shaft for delivery of the vapor through the tip.

18. The vapor delivery device of claim 16, wherein the vapor in the treatment volume is blocked from propagating beyond the occlusion location by the at least partially occluded venous vasculature at the occlusion layer.

19. The vapor delivery device of claim 16, wherein the treatment volume includes a length of the vein between the occlusion location and the tip of the vapor delivery shaft of about 1 cm to about 10 cm.

20. The vapor delivery device of claim 16, wherein the vapor delivery shaft is configured to be pulled peripherally along the vein to deliver the vapor into a different treatment volume of the vein that is defined at least in part by a different occlusion location.
